SEQUENCE LISTING (59 sequences)

SEQ ID NO: 1 - DNA, 4 bp, Artificial Sequence
  construct for recombinant expression
ttac

SEQ ID NO: 2 - DNA, 29 bp, Artificial Sequence
  construct for recombinant expression
aaagcccctagcttttagctatggggata

SEQ ID NO: 3 - DNA, 6 bp, Artificial Sequence
  construct for recombinant expression
attacc

SEQ ID NO: 4 - DNA, 100 bp, Artificial Sequence
  construct for recombinant expression
cgggctgcaggaattcgatttgcgctagtgcaaaaattaccaaaactaacgccttaaagcccctagcttttagctatggggatacaaggcgaaacgcctt

SEQ ID NO: 5 - DNA, 100 bp, Artificial Sequence
  construct for recombinant expression
cgggctgcaggaattcgatttgcgctagtgcaaaaattaccaaaactaacgccttaaagcccctagcttttagctatggggttcgcccggaaaacgcctt

SEQ ID NO: 6 - DNA, 56 bp, Artificial Sequence
  construct for recombinant expression
ctcatgctttagctagaatcccctagctttagctatggggagtatgtcaacaaatt

SEQ ID NO: 7 - DNA, 110 bp, Artificial Sequence
  construct for recombinant expression
ctcatgctttagctagaatcccctagctttagctatggggagtatgtcaacaaaactaacgccttaaagcccctagcttttagctatggggatacaaggcgaaacgcctt

SEQ ID NO: 8 - DNA, 110 bp, Artificial Sequence
  construct for recombinant expression
ctcatgctttagctagaatcccctagctttagctatggggagtatgtcaacaaaactaacgccttaaagcccctagcttttagctatggggatacaaggcgaaataaagg

SEQ ID NO: 9 - DNA, 110 bp, Artificial Sequence
  construct for recombinant expression
ctcatgctttagctagaatcccctagctttagctatggggagtatgtcaacaaaactaacgccttaaagcccctagcttttagctatggggttacaaggcgaaataaagg

SEQ ID NO: 10 - DNA, 110 bp, Artificial Sequence
  construct for recombinant expression
ctcatgctttagctagaatcccctagctttagctatggggagtatgtcaacaaaactaacgccttaaagcccctagcttttagctatggggttcgcccggaaaacgcctt

SEQ ID NO: 11 - DNA, 110 bp, Artificial Sequence
  construct for recombinant expression
ctcatgctttagctagaatcccctagctttagctatggggagtatgtcaacaaaactaacgccttaaagcccctagcttttagctatggggttcgcccggaaaatccggg

SEQ ID NO: 12 - DNA, 110 bp, Artificial Sequence
  construct for recombinant expression
ctcatgctttagctagaatcccctagctttagctatggggagtatgtcaacaaaactaacgccttaaagcccctagcttttagctatggggttgcccgaacaaacgcctt

SEQ ID NO: 13 - DNA, 110 bp, Artificial Sequence
  construct for recombinant expression
ctcatgctttagctagaatcccctagctttagctatggggagtatgtcaacaaaactaacgccttaaagcccctagcttttagctatggggttcgcccggatgattcctt

SEQ ID NO: 14 - DNA, 110 bp, Artificial Sequence
  construct for recombinant expression
ctcatgctttagctagaatcccctagctttagctatggggagtatgtcaacaaaactaacgccttaaaccccctagcttttagctatggggttcgcccggaaaacgcctt

SEQ ID NO: 15 - DNA, 110 bp, Artificial Sequence
  construct for recombinant expression
ctcatgctttagctagaatcccctagctttagctatggggagtatgtcaacaaaactaacgccttcaaacccctagcttttagctatggggttcgcccggaaaacgcctt

SEQ ID NO: 16 - DNA, 67 bp, Artificial Sequence
  construct for recombinant expression
ccgatgatgaggaacccccccctagagcttttattactgatgatgaggaacccccccctagtgatga

SEQ ID NO: 17 - DNA, 75 bp, Artificial Sequence
  construct for recombinant expression
ccgatgatgaggaacccccccccgccttgtttattactgatgatgaggaacccccccctagtgatgatgaggatt

SEQ ID NO: 18 - DNA, 75 bp, Artificial Sequence
  construct for recombinant expression
ccgatgatgaggaacccccccctccgggcgtcgttactgatgatgaggaacccccccctagtgatgatgaggatt

SEQ ID NO: 19 - DNA, 75 bp, Artificial Sequence
  construct for recombinant expression
ccgatgatgaggaaccccccccgttcgggctgattactgatgatgaggaacccccccctagtgatgatgaggatt

SEQ ID NO: 20 - DNA, 75 bp, Artificial Sequence
  construct for recombinant expression
ccgatgatgaggaacccaatcatccgggcgtcgttactgatgatgaggaacccccccctagtgatgatgaggatt

SEQ ID NO: 21 - DNA, 62 bp, Artificial Sequence
  construct for recombinant expression
ccgatgatgaggaacccccccctaggggcgtcgttactgatgatgaggaacccccccctagt

SEQ ID NO: 22 - DNA, 75 bp, Artificial Sequence
  construct for recombinant expression
ccgatgatgaggaacccccccctccgggcgtcgttagtgatgatgaggaacccccccctagtgatgatgaggatt

SEQ ID NO: 23 - DNA, 75 bp, Artificial Sequence
  construct for recombinant expression
ccgatgatgaggaacccccccctccgggcgtcgtgattgatgatgaggaacccccccctagtgatgatgaggatt

SEQ ID NO: 24 - DNA, 67 bp, Artificial Sequence
  construct for recombinant expression
ccgatgatgaggaacccccccctagagcttttattagtgatgatgaggaacccccccctagtgatga

SEQ ID NO: 25 - DNA, 67 bp, Artificial Sequence
  construct for recombinant expression
ccgatgatgaggaacccccccctagagcttttatgattgatgatgaggaacccccccctagtgatga

SEQ ID NO: 26 - DNA, 4 bp, Artificial Sequence
  construct for recombinant expression
tcaa

SEQ ID NO: 27 - DNA, 4 bp, Artificial Sequence
  construct for recombinant expression
ttac

SEQ ID NO: 28 - DNA, 4 bp, Artificial Sequence
  construct for recombinant expression
gaat

SEQ ID NO: 29 - DNA, 4 bp, Artificial Sequence
  construct for recombinant expression
aaag

SEQ ID NO: 30 - DNA, 4 bp, Artificial Sequence
  construct for recombinant expression
aaac

SEQ ID NO: 31 - DNA, 4 bp, Artificial Sequence
  construct for recombinant expression
caaa

SEQ ID NO: 32 - DNA, 10 bp, Artificial Sequence
  construct for recombinant expression
ttcgcccgga

SEQ ID NO: 33 - DNA, 15 bp, Artificial Sequence
  construct for recombinant expression
tccgggcgtcgttac

SEQ ID NO: 34 - DNA, 31 bp, Artificial Sequence
  construct for recombinant expression
gttcgggctgattacctgacactgggcctgc

SEQ ID NO: 35 - DNA, 31 bp, Artificial Sequence
  construct for recombinant expression
gtggcggaaaattacccgcaggctggtatca

SEQ ID NO: 36 - DNA, 31 bp, Artificial Sequence
  construct for recombinant expression
tcccgtgaactttacccggtggtgcatatcg

SEQ ID NO: 37 - DNA, 31 bp, Artificial Sequence
  construct for recombinant expression
tgacggtccttttacccgcaaacatgccgaa

SEQ ID NO: 38 - DNA, 31 bp, Artificial Sequence
  construct for recombinant expression
aatcacacagattacccgtaaacagcctgaa

SEQ ID NO: 39 - DNA, 31 bp, Artificial Sequence
  construct for recombinant expression
aggctgcgcagttaccgggtatatataagat

SEQ ID NO: 40 - DNA, 31 bp, Artificial Sequence
  construct for recombinant expression
aaaatacctggttacccaggccgtgccggca

SEQ ID NO: 41 - DNA, 31 bp, Artificial Sequence
  construct for recombinant expression
ctgggtgatatttacctgaatcataaataca

SEQ ID NO: 42 - DNA, 31 bp, Artificial Sequence
  construct for recombinant expression
tggcaatggtgttactgaacgcagccgtcag

SEQ ID NO: 43 - DNA, 31 bp, Artificial Sequence
  construct for recombinant expression
acgtccacgccttacgaatccctgcttgtaa

SEQ ID NO: 44 - DNA, 31 bp, Artificial Sequence
  construct for recombinant expression
gcgaatgctgtttacggggttttttactggt

SEQ ID NO: 45 - DNA, 31 bp, Artificial Sequence
  construct for recombinant expression
ccatccgtcctttacggtggtttctgagcag

SEQ ID NO: 46 - DNA, 31 bp, Artificial Sequence
  construct for recombinant expression
tccgggcgtcgttacagggggccagtatcac

SEQ ID NO: 47 - DNA, 31 bp, Artificial Sequence
  construct for recombinant expression
tggcaatggtgttacagggggccagtatcac

SEQ ID NO: 48 - DNA, 31 bp, Artificial Sequence
  construct for recombinant expression
acgtccactgattacgaatccctgcttgtaa

SEQ ID NO: 49 - DNA, 31 bp, Artificial Sequence
  construct for recombinant expression
acgtccacgccttacctgacactgggcctgc

SEQ ID NO: 50 - DNA, 31 bp, Artificial Sequence
  construct for recombinant expression
acgtccactgattacctgacactgggcctgc

SEQ ID NO: 51 - DNA, 31 bp, Artificial Sequence
  construct for recombinant expression
ctgggtgatatttacatgaatcataaataca

SEQ ID NO: 52 - DNA, 31 bp, Artificial Sequence
  construct for recombinant expression
ctgggtgatatttacgtgaatcataaataca

SEQ ID NO: 53 - DNA, 31 bp, Artificial Sequence
  construct for recombinant expression
ctgggtgatatttacttgaatcataaataca

SEQ ID NO: 54 - DNA, 31 bp, Artificial Sequence
  construct for recombinant expression
tggcaatggtgttaccgaacgcagccgtcag

SEQ ID NO: 55 - DNA, 31 bp, Artificial Sequence
  construct for recombinant expression
tggcaatggtgttaccgggggccagtatcac

SEQ ID NO: 56 - DNA, 31 bp, Artificial Sequence
  construct for recombinant expression
tggcaatggtgttacggggggccagtatcac

SEQ ID NO: 57 - DNA, 31 bp, Artificial Sequence
  construct for recombinant expression
tggcaatggtgttactgggggccagtatcac

SEQ ID NO: 58 - DNA, 26 bp, Artificial Sequence
  construct for recombinant expression
  misc_feature (1)..(2): n is a, c, g, or t
nnttaccaaaactaacgccttaaagc

SEQ ID NO: 59 - DNA, 66 bp, Artificial Sequence
  construct for recombinant expression
aattaccaaaactaacgccttaaagcccctagcttttagctatggggatacaaggcgaaacgcctt

(19) United States
(12) Patent Application Publication
(10) Pub. No.: US 2022/0145323 A1
(43) Pub. Date: May 12, 2022

(54) IMPROVED TRANSPOSON INSERTION SITES AND USES THEREOF

(71) Applicant: EUROPEAN MOLECULAR BIOLOGY LABORATORY, HEIDELBERG (DE)

(72) Inventors: ORSOLYA BARABAS, HEIDELBERG (DE); FREDERICK DYDA, BETHESDA, MD (US); ALISON BURGESS HICKMAN, BETHESDA, MD (US)

(21) Appl. No.: 17/602,126

(22) PCT Filed: Apr. 9, 2019
(86) PCT No.: PCT/EP2019/058916

§ 371 Date: Oct. 7, 2021

Related U.S. Application Data
(60) Provisional application No. 62/830,658, filed on Apr. 8, 2019.

(51) Int. Cl. C12N 15/85; C12N 15/90

(52) U.S. Cl. CPC C12N 15/85; C12N 2800/90; C12N 15/90

(57) ABSTRACT

The present invention relates to novel transposon constructs and uses thereof. The novel transposon constructs of this invention have been developed based on structure-guided engineering approaches of the IS608 transposon. Provided are polynucleotides encoding for transposon ends, which may advantageously be used for site-specific insertion of a nucleotide sequence of interest into the genome of a target cell or a target DNA molecule. The invention provides further nucleic acids, vectors and recombinant cells encoding or containing the improved polynucleotides encoding for transposon ends, as well as a transposase system. Hence, the invention provides many tools for molecular genetic approaches for genome alteration, such as cloning strategies. Furthermore provided are medical and non-medical uses of the polynucleotides of the invention. The invention is in particular useful as a tool for gene delivery in genetically modified cell based therapeutic approaches for treating various diseases and for genetic tagging of endogenous proteins in research.

### FIELD OF THE INVENTION

The present invention relates to novel transposon constructs and uses thereof. The novel transposon constructs of this invention have been developed based on structure-guided engineering approaches of the IS6o8 transposon. Provided are polynucleotides encoding for transposon ends, which may advantageously be used for site-specific insertion of a nucleotide sequence of interest into the genome of a target cell or a target DNA molecule. The invention provides further nucleic acids, vectors and recombinant cells encoding or containing the improved polynucleotides encoding for transposon ends, as well as a transposase system. Hence, the invention provides many tools for molecular genetic approaches for genome alteration, such as cloning strategies. Furthermore provided are medical and non-medical uses of the polynucleotides of the invention. The invention is in particular useful as a tool for gene delivery in genetically modified cell based therapeutic approaches for treating various diseases and for genetic tagging of endogenous proteins in research.

### DESCRIPTION

Transposable elements (TEs) are a large, ubiquitous group of mobile genetic elements that can autonomously move from one genomic location to another. They have had a dynamic role in genome remodelling and evolution, and most eukaryotic and prokaryotic genomes are rich in TE-related sequences. While most of these represent inactive TE remnants, various elements can still move, causing diverse adaptive or adverse phenotypes throughout the tree-of-life. For example, in bacteria TE mobilization has been linked to environmental adaptation and the emergence of multi-drug resistant pathogens. DNA transposons are discrete genetic entities ubiquitously spread across the tree of life that can move within and between genomes. They are prominent evolutionary forces fostering genome remodeling, evolutionary changes, transmission of antibiotic resistance determinants, and the development of new biological functions such as adaptive immunity.

Due to their inherent ability to carry and integrate DNA into foreign genomes, transposons provide widely used tools for genetic engineering. They have been successfully used for insertional mutagenesis allowing for example the characterization of gene functions and the identification of oncogenes and tumour suppressors. Moreover, they are also applied in transgenesis, providing efficient non-viral gene delivery vehicles that are now used in human gene therapy applications.

Conventionally, applied transposon systems consist of a transposon, made up of a gene of interest (genetic cargo) flanked by specific transposon end DNA sequences, and the transposase protein expressed from a separate plasmid or locus. The transposase specifically binds to the transposon DNA ends, cuts the transposon from a donor locus and integrates it in a new genomic location.

Transposons are now also applied in a number of clinical trials, some of which aim to ex vivo modify T cells by incorporating a chimeric antigen receptor (CAR) against malignancy-specific antigens. In these studies, the transposase inserts a CAR gene-carrying transposon from a donor plasmid into the genome of patient-derived T cells, which are successively re-infused in the cancer patient. The introduced CARs provide the T cells with new specificities to distinctively target the cancer cells and trigger effector functions upon antigen encounter. This therapy has shown unprecedented response rates (70%-90%) in the treatment of acute and chronic leukemia and will likely enter mainstream care for many B cell malignancies in the next years.

One of the simplest and best characterized transposons is the bacterial insertion sequence (IS) IS6o8 from Helicobacter pylori, a member of the IS200/IS605 family. Interestingly, the IS200/IS605 elements do not have inverted sequences at their ends characteristic of many other prokaryotic and eukaryotic transposons. Rather, imperfect palindromic (IP) sequences are located close to the transposon ends. IS6o8 exhibits an unusual transposition mechanism using single-stranded DNA (ssDNA) intermediates and integrates specifically at 4 nt target sequences in the genome. Transposon excision and integration is catalyzed by the element-encoded transposase TnpA, which belongs to the HUH (histidine-hydrophobic-histidine) endonuclease superfamily and uses a single catalytic tyrosine to cleave DNA. Previous crystallographic and biochemical studies of IS6o8 have shown that TnpA binds specifically to sub-terminal imperfect palindrome (IP) structures formed on the top strand of the left (LE) and right (RE) IS ends (named IPL and IPR, respectively).

The catalytic site of IS6o8 is assembled in trans within a protein dimer, with the catalytic tyrosine (located on the most C-terminal helix αD) contributed by one monomer and the HUH motif by the other. IS6o8 insertion occurs precisely 3′ to a specific TTAC tetranucleotide sequence that is then retained in the left transposon flank and is required for excision and subsequent transposition to a new site. Notably, the cleavage site sequences at LE and RE (CL and CR, respectively) are not directly recognized by TnpA, but form a complex set of base pairs with a tetranucleotide ‘guide’ sequence (GL or GR) located 5′ to the base of each IP hairpin. These interactions help to structure the nucleoprotein complex and activate transposon excision, creating a circular junction intermediate and simultaneously sealing the flanking donor DNA backbone.

After transposon end cleavage (i), the donor DNA sequence is precisely sealed and a circular transposon junction is formed (ii) as an intermediate before cleavage and re-integration into a new target site (iii and iv). Black wedges mark the positions of cleavage at the transposon ends (i) and 3′ to a target cleavage site (CT) (iii). Specific base-pairing between guide and cleavage sequences in the transposon before excision (i), and between GL and CT before re-integration (iii), are indicated with dotted lines. For integration, the TnpA-bound transposon junction specifically interacts with an ssDNA target by base pairing between GL and the TTAC target sequence (CT). This unique mode of target recognition by base pairing between transposon and target sequences provides an intriguing opportunity to redirect transposon integration to different sequences in a predictable way by only modifying GL in the transposon, as demonstrated previously in vitro and in vivo.

TEs are efficient DNA carriers and thus important tools for transgenesis and insertional mutagenesis. However, a major constraint of these tools in transgenesis is the very low specificity of their target site selection (e.g. the most used Sleeping Beauty and PiggyBac transposons integrate at specific di- or tetranucleotide sequences, respectively), which leads to integration at very diverse positions throughout the recipient genome. This poor target sequence specificity constitutes an important limitation for site-directed applications. Therefore, much effort has been dedicated to unravel the molecular basis of target DNA selection of a variety of TEs in order to design strategies to direct their integration to specific genomic sites.

### BRIEF DESCRIPTION OF THE INVENTION

Generally, and by way of brief description, the main aspects of the present invention can be described as follows:

In a first aspect, the invention pertains to A polynucleotide comprising a nucleic acid sequence encoding for, or being, at least one transposon end, wherein said at least one transposon end has the following structure in 5′ to 3′ direction: a 5′ flanking region, a transposase recognition site, such as a 5′ guide sequence, optionally a cloning cassette for introducing a genetic cargo construct, and a 3′ flanking region; characterized in that the 5′ flanking region and/or the 3′ flanking region comprise a target complementarity region having a sequence identity of at least 6o % to a nucleic acid sequence in a target genome.

In a second aspect, the invention pertains to a composition comprising the polynucleotide according to the first aspect of the invention, and a nucleic acid encoding an integrating enzyme, optionally wherein said nucleic acid is under the control of a promoter element, or an integrating polypeptide, such as an integrating enzyme polypeptide.

In a third aspect, the invention pertains to and expression construct comprising an expressible polynucleotide according to the first aspect, or a composition according to the second aspect, and a promoter element, wherein the promoter element is operably linked to the expressible polynucleotide to allow for the expression of the polynucleotide

In a fourth aspect, the invention pertains to a nucleic acid vector, comprising any compounds and/or compositions of the other aspects of the invention.

In a fifth aspect, the invention pertains to a recombinant cell, comprising any compounds and/or compositions of the other aspects of the invention.

In a sixth aspect, the invention pertains to a transposon system comprising
(a) any compounds and/or compositions of the other aspects of the invention; and
(b) a transposase polypeptide of the IS200/IS605 family, such as IS608, or a IS630, IS701, IS607, IS982, IS3, IS1, IS6, IS5, IS4, or IS66, preferably wherein said transposase polypeptide is the IS6o8-encoded transposase TnpA.

In a seventh aspect, the invention pertains to a method for gene delivery into a target cell comprising the following steps:
(a) bringing into contact the transposon system according to the sixth aspect of the invention with a target cell; and
(b) culturing said target cell under conditions permissive to the culture of said target cell.

In an eighth aspect, the invention pertains to a pharmaceutical composition, comprising any compounds and/or compositions of the other aspects of the invention, together with a pharmaceutically acceptable carrier and/or excipient.

In a ninth aspect, the invention pertains to a kit comprising
(a) any compounds and/or compositions of the other aspects of the invention; and/or
(b) a transposase polypeptide of the IS200/IS605 family, such as IS6o8, or a IS630, IS701, IS607, IS982, IS3, IS1, IS6, IS5, IS4, or IS66 transposase, preferably wherein said transposase polypeptide is the IS6o8-encoded transposase TnpA.

In a tenth aspect, the invention pertains to method of preventing and/or treating an infectious and/or a proliferative disease in a subject using any of the compounds or compositions of the invention.

### DETAILED DESCRIPTION OF THE INVENTION

[21] In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

The above problem is solved in a first aspect by a structure-based transposon engineering approach. The inventors present the crystal structure of IS6o8 TnpA in a ternary complex with transposon left end DNA (including the imperfect palindrome structure formed on the top strand of the left end (IPL) with the left guide sequence (GL) and 3 additional nucleotides downstream of IPL) and a target substrate (T6′) spanning the cleavage site (CT). The structure reveals the IS6o8 target capture complex in an active pre-cleavage state with uncleaved target DNA. The crystal structure provides novel insights into the recognition of the nucleotides surrounding the core 4 nt target recognition sequence. Specifically, it shows that target recognition involves base triplet interactions between GL, the 3′ flank of IPL and the target sequence.

Based on the structural insights from the crystal structure of the IS6o8 target capture complex as identified, the above problem is further solved by the design of novel transposon variants that create an extended set of specific base interactions with the target DNA, thereby recognizing longer target sites with high specificity. The inventors engineered IS6o8 variants to direct their integration specifically to various 12/17-nt long target sites by extending the base pair interaction network between the transposon and the target DNA. The inventors demonstrate in vitro that the engineered transposons efficiently select their intended target sites, providing a novel strategy and proof-of concept for targeting specific user-defined DNA sites.

This invention elucidates how the target choice of the insertion sequence IS6o8 from Helicobacter pylori can be re-programmed by changes in the transposon DNA. The strategy of this invention enables efficient targeting of unique DNA sequences with high specificity in an easily programmable manner, opening endless possibilities for the use of the transposon systems for site-specific gene insertions.

The above problem is solved in a first aspect by a polynucleotide comprising a nucleic acid sequence encoding for at least one transposon end, wherein said nucleic acid sequence has the following structure in 5′ to 3′ direction: a 5′ flanking region, a transposase recognition site, and a 3′ flanking region; characterized in that the 5′ flanking region and/or the 3′ flanking region comprise a target complementarity region having a sequence identity of at least 6o % to a nucleic acid sequence in a target genome, preferably at least 70%, more preferably at least 80%, even more preferably at least 90%, even more preferably at least 99%, and most preferably having a sequence identity of 100% to said target genome.

As used herein, the term “transposon end” or “terminal sequence” shall refer to a sequence located at one end of a transposon unit that can be cleaved by a transposase polypeptide when used in combination with a complementary sequence that is located at the opposing end of the vector or transposon unit. The pair of transposon ends is involved in the transposition activity of the transposon unit of the present disclosure, in particular involved in DNA addition or removal and excision and integration of DNA of interest. In one example, at least one pair of transposon ends appears to be the minimum sequence required for transposition activity in a plasmid. As would be understood by the person skilled in the art, to facilitate ease of cloning, the necessary terminal sequence may be as short as possible and thus contain as little inverted repeats as possible. Thus, in one example, the transposon unit of the present disclosure may comprise not more than one, not more than two, not more than three or not more than four pairs of inverted terminal repeats. In one example, the transposon unit of the present disclosure may comprise only one pair of transposon ends. Whilst not wishing to be bound by theory, it is envisaged that having more than one pair of transposon ends may be disadvantageous as it may lead to non-specific transposase binding to the multiple transposon ends and resulting in the removal of desired sequence or insertion of undesirable sequences.

Herein, the term “transposon unit” shall refer to at least one nucleic acid construct that encodes for two transposon ends, or to two or more nucleic acid constructs that encode for two transposon ends, and a cargo sequence that shall be introduced into a target cell genome or target nucleotide, such as a plasmid. Usually a transposon unit will be nucleic acid and may be a vector of any form suitable for transposition.

Said target cell can be a eukaryotic or a prokaryotic cell, such as a bacterial cell, a yeast cell, or a cell of a mammal, for example a cell of a human, a mouse, a rat, a rabbit, a dog, a monkey, or a cat. Target cells that are preferably used in context of this invention are also stem cells, such as, for example, embryonic, or adult stem cells, such as hematopoietic stem cells. A preferred target cell according to this invention is also a T-cell, a B-Cell, or a Chinese Hamster Ovary (CHO) cell.

As used herein, the terms “identical” or percent “identity”, when used anywhere herein in the context of two or more nucleic acid or protein/polypeptide sequences, refer to two or more sequences or subsequences that are the same or have (or have at least) a specified percentage of amino acid residues or nucleotides that are the same (i.e., at, or at least, about 60% identity, preferably at, or at least, 65%, 70%, 75%, 80%, 85%, 90%, 91%, 92%, 93% or 94%, identity, and more preferably at, or at least, about 95%, 96%, 97%, 98%, 99%, or higher identity over a specified region—preferably over their full length sequences—when compared and aligned for maximum correspondence over the comparison window or designated region) as measured using a sequence comparison algorithms, or by manual alignment and visual inspection (see, e.g., NCBI web site). In a particular embodiment, for example when comparing the protein or nucleic acid sequence of the transposase of the invention to for example a reference, the percentage identity can be determined by the Blast searches provided in NCBI; in particular for amino acid identity, those using BLASTP 2.2.28+ with the following parameters: Matrix: BLOSUM62; Gap Penalties: Existence: 11, Extension: 1; Neighboring words threshold: 11; Window for multiple hits: 40.

The term “transposase” as used herein refers to an enzyme that is a component of a functional nucleic acid-protein complex capable of transposition and which is mediating transposition. The term “transposase” also refers to integrases from retrotransposons or of retroviral origin. A “transposition reaction” as used herein refers to a reaction where a transposon inserts into a target nucleic acid. Primary components in a transposition reaction are a transposon and a transposase or an integrase enzyme. For example, the transposon system according to the invention preferably comprises the bacterial insertion sequence IS6o8 from Helicobacter pylori, which encodes the transposase TnpA.

In preferred embodiments of the present invention the polynucleotide is RNA, DNA, cDNA, PNA, or a combination thereof.

In some embodiments it is preferred that the transposase recognition site comprises imperfect palindrome sequences, inverted terminal repeats (ITRs), and/or direct terminal repeats (DTRs), preferably wherein said transposase recognition site comprises imperfect palindrome sequences.

In some embodiments the polynucleotide according to the invention is preferably a polynucleotide, wherein the polynucleotide further comprises in 5′ to 3′direction:
(a) Optionally, a 5′-cleavage site,
(b) a 5′-guide sequence,
(c) one or more 5′—transposon structural element(s),
(d) optionally, the cloning cassette for introducing a genetic cargo construct,
(e) a 3′ guide sequence,
(f) One or more 3′ transposon structural element(s),
(g) Optionally, a 3′-cleavage site.

In some embodiments of the invention the target complementarity region is in close sequence proximity to the 5′-guide sequence, preferably not more than 100 nucleotides apart, more preferably not more than 5o nucleotides apart, most preferably between 15 to 40 (or 20 to 30) nucleotides apart from the 3′end of 5′ guide sequence.

In some embodiments of the invention the target complementarity region is located 3′ of the first 5′ transposon structural element, more preferably, between two 5′ transposon structural elements.

In some embodiments of the invention the 5′ and/or 3′ transposon structural element is a sequence, that is capable of forming a 3-dimensional structure, such as a hairpin, within the polynucleotide and preferably is selected from imperfect or perfect palindrome sequences, inverted terminal repeats (ITRs), and/or direct terminal repeats (DTRs), most preferably wherein said transposon structural element is an imperfect palindrome sequence.

$$\begin{matrix}In some emodiments of the invention the polynucleotide is preferred if & \\ The 5 ’ \text{-} cleavage site has the sequence TTAC ; and / or & ( a ) \\ The 3 ’ \text{-} cleavage site has the sequence TCAA ; and / or & ( b ) \\ The 5 ’ \text{-} guide sequence has the sequence AAAG ; and / or & ( c ) \\ The 3 ’ \text{-} guide sequence has the sequence GAAT ; & ( d )\end{matrix}$$

optionally, with not more than one, preferably none, nucleotide variation of these sequences.

In some embodiments of the invention the polynucleotide of the invention does not comprise a sequence encoding for a functional transposase protein, preferably not comprising tnpA and/or tnpB genes.

In some embodiments of the invention the cloning cassette comprises one or more restriction enzyme recognition sites.

In some embodiments of the invention the target complementarity region comprises a sequence complementary to a region within a target genome, which (i) is located outside of an expressible genetic element, or (ii) within an expressible genetic element, and preferably wherein (ii) the target complementarity region is selected such that the expressible genetic element is not expressible, or reduced expressible, or increased expressible and/or the expressed sequence is dis-functional after integration of the transposon.

In some embodiments of the invention the at least one transposon end is not capable of being mobilized within a target genome.

In addition, in some embodiments, the transposon comprises a single-strand transposon, and/or a double-strand transposon, preferably wherein said transposon comprises a single-strand transposon.

A further preferred embodiment then relates to the polynucleotide according to this invention, wherein the target complementarity region in the s′ flanking region and/or the 3′ flanking region of the polynucleotide is 1 to 100 nucleotides long, more preferably 2 to 50, more preferably 4 to 20, and most preferably 8 to 13 nucleotides long.

Further preferred is a polynucleotide according to this invention, wherein no nucleic acid sequence is located between said target complementarity region and said transposase recognition site or wherein a nucleic acid sequence is located between said target complementarity region and said transposase recognition site, preferably wherein said sequence is between 1 and 100 nucleotides long, more preferably between 1 and 10 nucleotides long, even more preferably between 1 and 5 nucleotides long, and most preferably wherein said sequence is 2 nucleotides long.

A further preferred embodiment relates to the polynucleotide according to this invention, wherein said target complementarity region is located in the 3′ flanking region, preferably wherein said target complementarity region is located at the 5′ end of said 3′ flanking region, or wherein said target complementarity region is located in the 5′ flanking region, preferably wherein said target complementarity region is located at the 3′ end of said 5′ flanking region.

The polynucleotide according to this invention is preferably for use in site-specific insertion of a nucleotide sequence of interest to be inserted into the genome of a target cell, or into another target DNA molecule, such as a plasmid of genomic DNA, for example a plasmid, such as a plasmid in a test tube. More preferably, said nucleotide sequence of interest is a modified transposon sequence, optionally wherein the transposase gene is replaced with a sequence of interest. The nucleotide sequence of interest is a cargo sequence to be inserted into the genome of a target cell.

Said target cell can be a eukaryotic or a prokaryotic cell, such as a bacterial cell, a yeast cell, or a cell of a mammal, for example a cell of a human, a mouse, a rat, a rabbit, a dog, a monkey, or a cat. Target cells that are preferably used in context of this invention are also stem cells such as, for example, embryonic, or adult stem cells, such as hematopoietic stem cells. A preferred target cell according to this invention is also a T-cell, a B-Cell, or a Chinese Hamster Ovary (CHO) cell.

In a preferred embodiment, the transposon sequence comprises an insertion sequence (IS) of the IS200/IS605 family, such as IS6o8, ISDra2, IS605, or IS630, IS701, IS607, IS982, IS3, IS1, IS6, IS5, IS4, or IS66, preferably wherein said insertion sequence (IS) is IS6o8.

Further preferred is that the transposase recognition site comprises the nucleic acid sequence of SEQ ID No: 61 (CCCCTAGCTTTTAGCTATGGGG). Further, the sequence TTAC (SEQ ID No: 1) is the preferred target cleavage site (cleavage target site, CT) of TnpA.

Another aspect of the invention pertains to a composition comprising the polynucleotide according to this invention, and a nucleic acid encoding an integrating enzyme, optionally wherein said nucleic acid is under the control of a promoter element, or an integrating polypeptide, such as an integrating enzyme polypeptide.

Further preferred is a composition, wherein the nucleic acid of said composition is DNA, cDNA, PNA, RNA, or a combination thereof, or an expression vector expressing said nucleic acid.

According to this invention, it is preferred that the polynucleotide encoding for said at least one transposon end and the nucleic acid encoding said integrating enzyme are the same nucleic acid molecules. Alternatively, it is also preferred that the polynucleotide encoding for said at least one transposon end and the nucleic acid encoding said integrating enzyme are separate nucleic acid molecules.

A further preferred embodiment relates to a composition, wherein the integrating enzyme is a transposase of the IS200/IS605 family, such as IS6o8, or an IS630, IS701, IS6o7, IS982, IS3, IS1, IS6, IS5, IS4, or IS66 transposase. Further preferred is that said integrating enzyme is a histidine-hydrophobic-histidine endonuclease, such as the IS6o8-encoded transposase TnpA.

In another aspect of the invention there is provided an expression construct, comprising an expressible polynucleotide according to this invention, or a composition according to this invention, and a promoter element, wherein the promoter element is operably linked to the expressible polynucleotide to allow for the expression of the polynucleotide.

Another aspect of the invention pertains to a nucleic acid vector comprising a polynucleotide according to this invention, or comprising a composition according to this invention, and/or comprising an expression construct according to this invention.

Yet another aspect of the invention relates to a recombinant cell comprising a polynucleotide according to this invention, or comprising a composition according to this invention, or comprising an expression construct according to this invention, and/or comprising a nucleic acid vector according to this invention.

The recombinant cell is preferably a cell suitable for recombinant protein expression, preferably for recombinant protein expression of the transposase polypeptide of the invention. The recombinant cell is a cell, such as a bacterial cell or eukaryotic cell, most preferably a bacterial cell such as E. coli or an insect cell, such as Drosophila S2 cell or a mammalian cell such as HEK293T cell.

A further aspect of the invention pertains a transposon system comprising
(a) a polynucleotide according to this invention, an expression construct according to this invention, a nucleic acid vector according to this invention, and/or a recombinant cell according to this invention; and
(b) a transposase polypeptide of the IS200/IS605 family, such as IS608, or of or IS630, IS701, IS6o7, IS982, IS3, IS1, IS6, IS5, IS4, and IS66, preferably wherein said transposase polypeptide is the IS6o8-encoded transposase TnpA.

Yet another aspect of the invention relates to the in vitro use of a transposon system according to this invention for gene delivery into a target cell, a target plasmid, and/or a target polynucleotide. The gene delivery is preferably an ex vivo gene delivery into a target cell, such as a target cell selected from a stem cell, such as a hematopoietic or embryonic stem cell, a T-cell, B-Cell or Chinese Hamster Ovary (CHO) cell. Such gene delivery into a target cell is particularly useful for cloning strategies or cloning purposes, such as for site-specific insertion of genetic cargo into nucleic molecules, such as into plasmid DNA. The use of the polynucleotide according to this invention enables the insertion of large genetic molecules into very specific locations in target DNA, such as plasmid DNA. Moreover, the insertion of the polynucleotide according to this invention allows the tagging of specific locations within the target nucleic molecule. The latter is useful for sequencing protocols, where the specific “barcoding”, or tagging of nucleic acid molecules is desirable.

An additional aspect of the invention pertains to an in vitro method for gene delivery into a target cell comprising the following steps:
(a) bringing into contact the transposon system according to this invention with a target cell; and
(b) culturing said target cell under conditions permissive to the culture of said target cell.

A further aspect of the invention then relates to a pharmaceutical composition comprising a polynucleotide according to this invention, a composition according to this invention, an expression construct according to this invention, a nucleic acid vector according to this invention, a recombinant cell according to this invention, and/or a transposon system according to this invention, together with a pharmaceutically acceptable carrier and/or excipient.

In another aspect, a pharmaceutical composition is provided, comprising a transposase polypeptide, a polynucleotide, a vector, and/or an expression construct, together with a pharmaceutically acceptable carrier and/or excipient.

Yet another aspect of the invention relates to a kit comprising
(a) a polynucleotide according to this invention, an expression construct according to this invention, a nucleic acid vector according to this invention, and/or a recombinant cell according to this invention; and
(b) a transposase polypeptide of the IS200/IS605 family, such as IS608, or a IS630, IS701, IS6o7, IS982, IS3, IS1, IS6, IS5, IS4, and IS66 transposase, preferably wherein said transposase polypeptide is the IS6o8-encoded transposase TnpA.

An additional aspect of the invention pertains to a kit comprising a polynucleotide according to this invention, a composition according to this invention, an expression construct according to this invention, a nucleic acid vector according to this invention, a recombinant cell according to this invention, or a transposon system according to this invention; and instructions for use.

The compounds and systems of the invention may preferably find application in medicine. Therefore, such compounds and systems of the invention are preferably for use in the treatment of a disease. Such diseases may be, for example, proliferative diseases, such as cancer. For a cancer treatment, the invention may be used in context of the generation of modified immune cells. For example, the invention can be used to introduce into immune cells T cell receptors (TCR) or CARs or other immune molecules, to strengthen and target a patient's immune system against cancer cells. Immune cells that can be modified may be selected from human T lymphocytes or B cells. Other diseases that could benefit from the invention are, for example, genetic disorders that are characterized by the loss of a gene function. In such diseases, cells could be modified with the invention to include a healthy copy of the disease associated gene. Other target cells that are preferably used in context of the invention are stem cells, such as, for example, embryonic, or adult stem cells, such as hematopoietic stem cells.

Another aspect of the invention relates to a compound for use in the treatment of a disease, wherein the compound is selected from the polynucleotide according to this invention, the composition according to this invention, the expression construct according to this invention, the nucleic acid vector according to this invention, the recombinant cell according to this invention, the transposon system according to this invention, the pharmaceutical composition according to this invention, and/or the kit according to this invention, preferably wherein the disease is an infectious and/or a proliferative disease, more preferably wherein said infectious disease is a bacterial infection, such as an H. pylori infection, and/or wherein said proliferative disease is cancer, such as non-small cell lung cancer, small cell lung cancer, renal cell cancer, brain cancer, gastric cancer, colorectal cancer, hepatocellular cancer, head and neck cancer, pancreatic cancer, prostate cancer, leukemia, breast cancer, Merkel cell carcinoma, melanoma, ovarian cancer, urinary bladder cancer, uterine cancer, gallbladder and bile duct cancer, esophageal cancer, or a combination thereof, preferably wherein said cancer is H. pylori-induced cancer.

Yet another aspect of the invention relates to the polynucleotide according to this invention, the composition according to this invention, the expression construct according to this invention, the nucleic acid vector according to this invention, the recombinant cell according to this invention, the transposon system according to this invention, the pharmaceutical composition according to this invention, and/or the kit according to this invention, for use in the diagnosis and/or treatment of an infectious and/or proliferative disease, or for use in the manufacture of a medicament against an infectious and/or proliferative disease, preferably wherein said infectious disease is a bacterial infection, such as an H. pylori infection, or wherein said proliferative disease is cancer, such as non-small cell lung cancer, small cell lung cancer, renal cell cancer, brain cancer, gastric cancer, colorectal cancer, hepatocellular cancer, head and neck cancer, pancreatic cancer, prostate cancer, leukemia, breast cancer, Merkel cell carcinoma, melanoma, ovarian cancer, urinary bladder cancer, uterine cancer, gallbladder and bile duct cancer, esophageal cancer, or a combination thereof, preferably wherein said cancer is H. pylori-induced cancer.

A further aspect of the invention relates to a method of preventing and/or treating an infectious and/or a proliferative disease in a subject, the method comprising administering to the subject an effective amount of a polynucleotide according to this invention, a composition according to this invention, an expression construct according to this invention, a nucleic acid vector according to this invention, a recombinant cell according to this invention, a transposon system according to this invention, and/or a pharmaceutical composition according to this invention, thereby preventing and/or treating said infectious and/or proliferative disease in the subject, preferably wherein said infectious disease is a bacterial infection, such as an H. pylori infection, or wherein said proliferative disease is cancer, such as non-small cell lung cancer, small cell lung cancer, renal cell cancer, brain cancer, gastric cancer, colorectal cancer, hepatocellular cancer, head and neck cancer, pancreatic cancer, prostate cancer, leukemia, breast cancer, Merkel cell carcinoma, melanoma, ovarian cancer, urinary bladder cancer, uterine cancer, gallbladder and bile duct cancer, esophageal cancer, or a combination thereof, preferably wherein said cancer is H. pylori-induced cancer.

Yet another aspect of the invention relates to the polynucleotide according to this invention, the composition according to this invention, the expression construct according to this invention, the nucleic acid vector according to this invention, the recombinant cell according to this invention, the transposon system according to this invention, the pharmaceutical composition according to this invention, and/or the kit according to this invention, for use in a gene delivery method, preferably wherein said method is for gene delivery into a cell selected from an eukaryotic and a prokaryotic cell, such as a cell of a human, a mouse, a rabbit, a dog, a monkey, a cat, a bacterium, or a yeast cell.

A further aspect of the invention relates to the use of the polynucleotide according to this invention for cloning strategies or cloning purposes, such as for site-specific insertion of genetic cargo into nucleic molecules, such as into plasmid DNA. The advantage of the polynucleotide according to this invention is that large genetic cargo can easily be inserted at very specific sites in target DNA, such as into plasmid DNA.

Yet another aspect of this invention relates to the use of the polynucleotide according to this invention for insertion into specific locations of a target nucleic molecule, wherein the inserted polynucleotide allows the tagging of said specific location within the target nucleic molecule. The latter is useful for sequencing protocols, where the specific “barcoding”, or tagging of nucleic acid molecules is desirable. Accordingly, the polynucleotide of this invention is for use in a broad range of applications, including many therapeutic uses and non-therapeutic uses.

Another aspect of the invention pertains to the polynucleotide according to this invention, the composition according to this invention, the expression construct according to this invention, the nucleic acid vector according to this invention, the recombinant cell according to this invention, the transposon system according to this invention, the pharmaceutical composition according to this invention, and/or the kit according to this invention, for use in manufacturing an engineered cell for a cell therapy, such as a T cell or a T cell progenitor, such as a CAR-T cell.

A preferred embodiment relates to said use in manufacturing an engineered cell for a cell therapy, preferably wherein the T cell or T cell progenitor is autologous, or wherein the T cell or T cell progenitor is allogeneic.

Yet another aspect of the invention relates to a method of treating cancer in a subject in need thereof, comprising:
(a) isolating a cell from said subject or from a healthy donor;
(b) transfecting or transforming the cell with a polynucleotide according to this invention, a composition according to this invention, an expression construct according to this invention, a nucleic acid vector according to this invention, and/or a transposon system according to this invention to produce a transfected or transformed cell;
(c) expanding the transfected or transformed cell to produce a plurality of transfected or transformed cells; and
(d) administering the plurality of transfected or transformed cells to said subject.

A preferred embodiment further relates to the method of treating cancer in a subject in need thereof, wherein the cell is a T cell or a T cell progenitor, optionally wherein said T cell or T cell progenitor is autologous or allogeneic.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, 15%, 10%, and for example 5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES AND SEQUENCES

The figures show:

FIG. 1: shows sequence hallmarks affecting IS6o8 target selection.

FIG. 2: shows that IS6o8 can be specifically targeted to longer integration sites by extended LE/target base pairing. (A) Close-up of the TnpA/LE29/T6′ structure, highlighting the proximity between the 3′ end of IPL and the 5′ end of the target oligonucleotide. (B) shows a schematic design of the IS608 transposon junction (Ji, where ‘i’ is a variable indicating a specific variant number) and complementary target substrates (Tic, with ‘i’ marking a specific variant as above) used for retargeting. (C) Sequencing DNA PAGE gel monitoring J1 cleavage and integration into its T1c complementary target. (D) J1 integrates selectively into its complementary target substrate (Tic) even in the excess of scrambled target substrates.

FIG. 3: shows the IS6o8 integration specificity can be enhanced to 17 nt sites and retargeted to non-native target sites.

The sequences show: [it might make sense to include the Cas9 protein sequence of the Cas9 protein used in the experiments]

(cleavage target site, CT)
SEQ ID No: 1
TTAC
(LE29)
SEQ ID NO: 2
AAAGCCCCTAGCTTTTAGCTATGGGGATA
(T6′ Activity Assays)
SEQ ID NO: 3
ATTACC
(LE)
SEQ ID NO: 4
CGGGCTGCAGGAATTCGATTTGCGCTAGTGCAAAAATTACCAAAACTA
ACGCCTTAAAGCCCCTAGCTTTTAGCTATGGGGATACAAGGCGAAACG
CCTT
(LE1)
SEQ ID NO: 5
CGGGCTGCAGGAATTCGATTTGCGCTAGTGCAAAAATTACCAAAACTA
ACGCCTTAAAGCCCCTAGCTTTTAGCTATGGGGTTCGCCCGGAAAACG
CCTT
(RE)
SEQ ID NO: 6
RE CTCATGCTTTAGCTAGAATCCCCTAGCTTTAGCTATGGGGAGTAT
GTCAACAAATT
(Jwt)
SEQ ID NO: 7
CTCATGCTTTAGCTAGAATCCCCTAGCTTTAGCTATGGGGAGTATGTC
AACAAAACTAACGCCTTAAAGCCCCTAGCTTTTAGCTATGGGGATACA
AGGCGAAACGCCTT
(Jwt-oh)
SEQ ID NO: 8
CTCATGCTTTAGCTAGAATCCCCTAGCTTTAGCTATGGGGAGTATGTC
AACAAAACTAACGCCTTAAAGCCCCTAGCTTTTAGCTATGGGGATACA
AGGCGAAATAAAGG
(Jwt-oh-42T)
SEQ ID NO: 9
CTCATGCTTTAGCTAGAATCCCCTAGCTTTAGCTATGGGGAGTATGTC
AACAAAACTAACGCCTTAAAGCCCCTAGCTTTTAGCTATGGGGTTACA
AGGCGAAATAAAGG
(J1)
SEQ ID NO: 10
CTCATGCTTTAGCTAGAATCCCCTAGCTTTAGCTATGGGGAGTATGTC
AACAAAACTAACGCCTTAAAGCCCCTAGCTTTTAGCTATGGGGTTCGC
CCGGAAAACGCCTT
(J1-h)
SEQ ID NO: 11
CTCATGCTTTAGCTAGAATCCCCTAGCTTTAGCTATGGGGAGTATGTC
AACAAAACTAACGCCTTAAAGCCCCTAGCTTTTAGCTATGGGGTTCGC
CCGGAAAATCCGGG
(J2)
SEQ ID NO: 12
CTCATGCTTTAGCTAGAATCCCCTAGCTTTAGCTATGGGGAGTATGTC
AACAAAACTAACGCCTTAAAGCCCCTAGCTTTTAGCTATGGGGTTGCC
CGAACAAACGCCTT
(J3)
SEQ ID NO: 13
CTCATGCTTTAGCTAGAATCCCCTAGCTTTAGCTATGGGGAGTATGTC
AACAAAACTAACGCCTTAAAGCCCCTAGCTTTTAGCTATGGGGTTCGC
CCGGATGATTCCTT
(J4)
SEQ ID NO: 14
CTCATGCTTTAGCTAGAATCCCCTAGCTTTAGCTATGGGGAGTATGTC
AACAAAACTAACGCCTTAAACCCCCTAGCTTTTAGCTATGGGGTTCGC
CCGGAAAACGCCTT
(J5)
SEQ ID NO: 15
CTCATGCTTTAGCTAGAATCCCCTAGCTTTAGCTATGGGGAGTATGTC
AACAAAACTAACGCCTTCAAACCCCTAGCTTTTAGCTATGGGGTTCGC
CCGGAAAACGCCTT
(Tr)
SEQ ID NO: 16
CCGATGATGAGGAACCCCCCCCTAGAGCTTTTATTACTGATGATGAGG
AACCCCCCCCTAGTGATGA
(Twtc)
SEQ ID NO: 17
CCGATGATGAGGAACCCCCCCCCGCCTTGTTTATTACTGATGATGAGG
AACCCCCCCCTAGTGATGATGAGGATT
(T1c)
SEQ ID NO: 18
CCGATGATGAGGAACCCCCCCCTCCGGGCGTCGTTACTGATGATGAGG
AACCCCCCCCTAGTGATGATGAGGATT
(T2c)
SEQ ID NO: 19
CCGATGATGAGGAACCCCCCCCGTTCGGGCTGATTACTGATGATGAGG
AACCCCCCCCTAGTGATGATGAGGATT
(T3c_1)
SEQ ID NO: 20
CCGATGATGAGGAACCCAATCATCCGGGCGTCGTTACTGATGATGAGG
AACCCCCCCCTAGTGATGATGAGGATT
(T3c_2)
SEQ ID NO: 21
CCGATGATGAGGAACCCCCCCCTAGGGGCGTCGTTACTGATGATGAGG
AACCCCCCCCTAGT
(T4c)
SEQ ID NO: 22
CCGATGATGAGGAACCCCCCCCTCCGGGCGTCGTTAGTGATGATGAGG
AACCCCCCCCTAGTGATGATGAGGATT
(T5c)
SEQ ID NO: 23
CCGATGATGAGGAACCCCCCCCTCCGGGCGTCGTGATTGATGATGAGG
AACCCCCCCCTAGTGATGATGAGGATT
(T4r)
SEQ ID NO: 24
CCGATGATGAGGAACCCCCCCCTAGAGCTTTTATTAGTGATGATGAGG
AACCCCCCCCTAGTGATGA
(T5r)
SEQ ID NO: 25
CCGATGATGAGGAACCCCCCCCTAGAGCTTTTATGATTGATGATGAGG
AACCCCCCCCTAGTGATGA
(right cleavage site, CR)
SEQ ID NO: 26
TCAA
(left cleavage site, CL)
SEQ ID NO: 27
TTAC
(right guide sequence, GR)
SEQ ID NO: 28
GAAT
(left guide sequence, GL)
SEQ ID NO: 29
AAAG
SEQ ID NO: 30
AAAC
SEQ ID NO: 31
CAAA
SEQ ID NO: 32
TTCGCCCGGA
SEQ ID NO: 33
TCCGGGCGTCGTTAC
(SET-1 ‘1.1’)
SEQ ID NO: 34
GTTCGGGCTGATTACCTGACACTGGGCCTGC
(SET-1 ‘1.2’)
SEQ ID NO: 35
GTGGCGGAAAATTACCCGCAGGCTGGTATCA
(SET-1 ‘1.3’)
SEQ ID NO: 36
TCCCGTGAACTTTACCCGGTGGTGCATATCG
(SET-1 ‘1.4’)
SEQ ID NO: 37
TGACGGTCCTTTTACCCGCAAACATGCCGAA
(SET-1 ‘1.5’)
SEQ ID NO: 38
AATCACACAGATTACCCGTAAACAGCCTGAA
(SET-1 ‘1.6’)
SEQ ID NO: 39
AGGCTGCGCAGTTACCGGGTATATATAAGAT
(SET-1 ‘1.7’)
SEQ ID NO: 40
AAAATACCTGGTTACCCAGGCCGTGCCGGCA
(SET-1 ‘1.8’)
SEQ ID NO: 41
CTGGGTGATATTTACCTGAATCATAAATACA
(SET-2 ‘2.1’)
SEQ ID NO: 42
TGGCAATGGTGTTACTGAACGCAGCCGTCAG
(SET-2 ‘2.2’)
SEQ ID NO: 43
ACGTCCACGCCTTACGAATCCCTGCTTGTAA
(SET-2 ‘2.3’)
SEQ ID NO: 44
GCGAATGCTGTTTACGGGGTTTTTTACTGGT
(SET-2 ‘2.4’)
SEQ ID NO: 45
CCATCCGTCCTTTACGGTGGTTTCTGAGCAG
(SET-2 ‘2.5’)
SEQ ID NO: 46
TCCGGGCGTCGTTACAGGGGGCCAGTATCAC
(SET-2 ‘2.6’)
SEQ ID NO: 47
TGGCAATGGTGTTACAGGGGGCCAGTATCAC
(SET-2 ‘2.2u’)
SEQ ID NO: 48
ACGTCCACTGATTACGAATCCCTGCTTGTAA
(SET-2 ‘2.2d’)
SEQ ID NO: 49
ACGTCCACGCCTTACCTGACACTGGGCCTGC
(SET-2 ‘2.2ud’)
SEQ ID NO: 50
ACGTCCACTGATTACCTGACACTGGGCCTGC
(‘1.8a’)
SEQ ID NO: 51
CTGGGTGATATTTACATGAATCATAAATACA
(‘1.8g’)
SEQ ID NO: 52
CTGGGTGATATTTACGTGAATCATAAATACA
(‘1.8t)
SEQ ID NO: 53
CTGGGTGATATTTACTTGAATCATAAATACA
(‘2.1c)
SEQ ID NO: 54
TGGCAATGGTGTTACCGAACGCAGCCGTCAG
(‘2.6c)
SEQ ID NO: 55
TGGCAATGGTGTTACCGGGGGCCAGTATCAC
(‘2.6g)
SEQ ID NO: 56
TGGCAATGGTGTTACGGGGGGCCAGTATCAC
(‘2.6t)
SEQ ID NO: 57
TGGCAATGGTGTTACTGGGGGCCAGTATCAC
SEQ ID NO: 58
NNTTACCAAAACTAACGCCTTAAAGC
SEQ ID NO: 59
AATTACCAAAACTAACGCCTTAAAGCCCCTAGCTTTTAGCTATGGGGA
TACAAGGCGAAACGCCTT
SEQ ID NO: 60
TTTAGCTAGAATCCCCTAGCTTTAGCTATGGGGAGTATGTCAANNNN
SEQ ID NO: 61
CCCCTAGCTTTTAGCTATGGGG

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

### The Examples Show

### Example 1: TnpA Prefers Target Sites with a C in Position+1

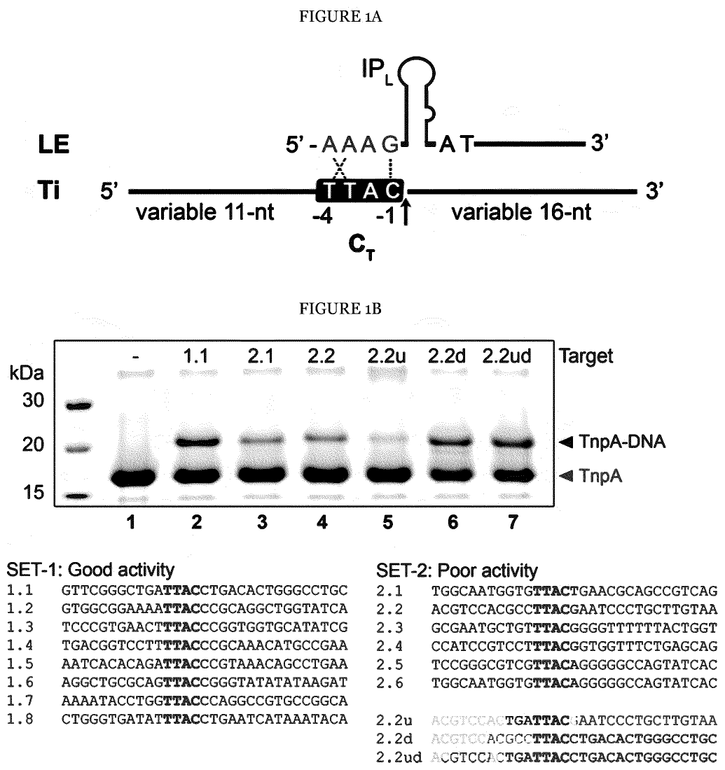

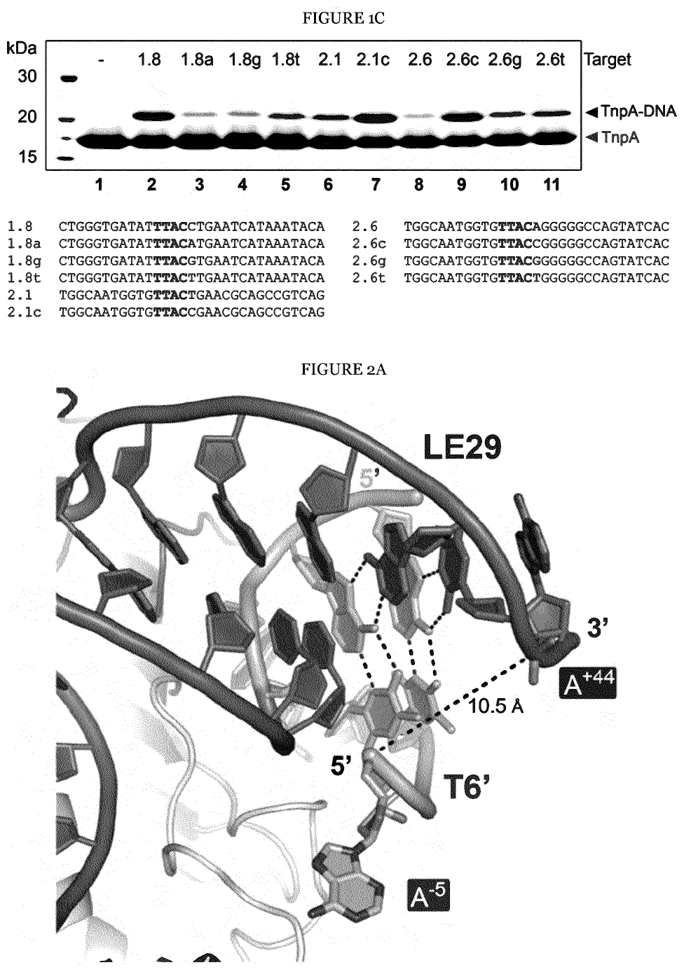

To determine whether the cleavage activity of TnpA is affected by the sequence surrounding the target cleavage site (TTAC, CT) and in particular by the identity of the nucleotide in position+1 downstream of the cleavage site, the inventors performed in vitro target cleavage assays with target oligonucleotides containing variable sequences at both sides of the TTAC sequence. FIG. 1(A) shows a scheme of the IS6o8 left end (LE) and target oligos (Ti) used to monitor TnpA mediated cleavage with variable sequences upstream and downstream of the core TTAC target sequence (CT). Arrow indicates the position of target cleavage.

In FIG. 1(B), cleavage assays monitoring covalent TnpA-DNA complex formation on SDS-PAGE gels are shown. For this, TnpA/LE complexes were incubated with different targets, and the cleavage activity was monitored by comparing the ratio of free TnpA and TnpA covalently bound to the 3′ flank of cleaved substrates in SDS-gels. Upon Ti cleavage, TnpA becomes covalently attached to the variable 16 nt sequence downstream of the cleavage position and can be resolved from unmodified TnpA. Based on the levels of covalent complexes formed, targets were classified as: SET-1, with good cleavage activity; and SET-2, with poor activity, as shown below the gel. Cleavage reactions are shown for representative SET-1 and SET-2 targets (lanes 2-4). The negative control (lane 1) does not contain target DNA. Cleavage reactions for derivatives of target 2.2, with the sequence upstream (u), downstream (d) of TTAC or both (ud) replaced by the corresponding sequence from target 1.1 (see sequences below SET-2) are shown in lanes 5-7. Remarkably, SET-1 contained only targets with a C in position+1, whereas SET-2 included other nucleotides. The inventors then replaced the sequence upstream and/or downstream of CT in a SET-2 representative oligo (2.2) with the corresponding sequence from an efficient SET-1 target (1.1). This showed that the upstream sequence had little effect on cleavage activity, whereas replacement of the sequence downstream of CT greatly increased cleavage, indicating its role in determining cleavage efficiency.

In FIG. 1(C), covalent complex formation is monitored on SDS-PAGE and target sequences are shown. To directly test the specific impact of C+1 on cleavage activity in particular, the inventors performed gain and loss-of-activity experiments by changing only the nucleotide in this position in target oligos from SET-1 (1.8) and SET-2 (2.1 and 2.6) (FIG. 1C). The results revealed that replacing C+1 with other nucleotides in a SET-1 target reduced TnpA cleavage activity, whereas introducing a Cin position+1 in a SET-2 target rescued cleavage, clearly showing that substrates with C in this position are better targets for TnpA.

### Example 2: IS6o8 Target Specificity can be Increased by Rational Design of Extended Base Pairing

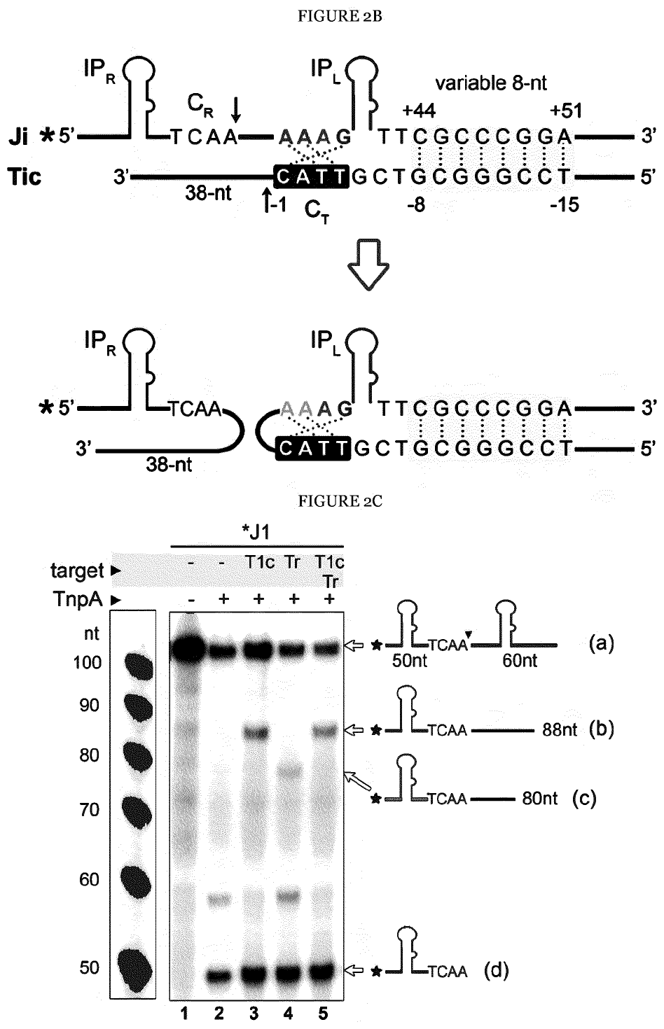

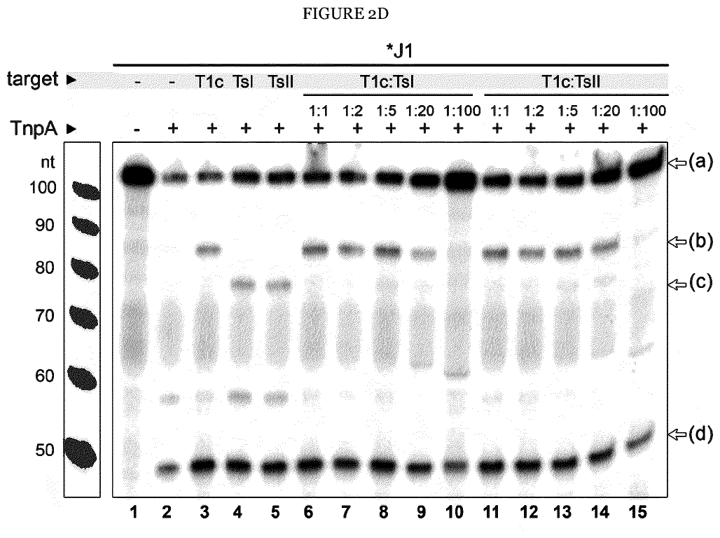

One remarkable feature of the TnpA/LE29/T6′ structure is that the 5′-end of T6′ is located near the 3′ base of the IPL stem loop in LE29. FIG. 2(A) shows a close-up of the TnpA/LE29/T6′ structure, highlighting the proximity between the 3′ end of IPL and the 5′ end of the target oligonucleotide. The distance between the 05′ oxygen atom of A-5 in T6′ and the phosphorous atom (P) of A+44 in LE29 is 10.5° A (dashed line). This suggested that introducing additional base pairing interactions at this transposon/target interface might provide a strategy for increasing target site specificity. Therefore, the inventors designed transposon sequences including specific 8 nt long sequences at positions+44 to +51 in LE and corresponding target substrates with a complementary 8 nt sequence upstream of CT. FIG. 2(B) shows a schematic design of the IS6o8 transposon junction (Ji, where ‘i’ is a variable indicating a specific variant number) and complementary target substrates (Tic, with ‘i’ marking a specific variant as above) used for retargeting. Each set of Ji/Tic oligos was designed to include an 8 bp complementary region between the 3′ extension of the IPL and the sequence upstream of the native TTAC target site (light blue shade). The 8 bp complementary sequence displayed here corresponds to the Ji/Tic pair. 32P radioisotope labeling is indicated by an asterisk. Upon target cleavage and integration (at the arrow), the radiolabeled 5′ segment of the junction upstream of the cleavage site (5o nt) is attached to the 3′ segment of the target (38 nt). The region of extended complementarity in the target was placed 3 nt apart from CT to provide flexibility for optimal interaction. The 3 nt linker size was chosen as it best supported TnpA cleavage in the initial tests of the inventors. Moreover, the triplet-forming A+42 base at the 3′ end of LE was mutated to T, to minimize steric constrains while maintaining the triplet interaction. The inventors then assayed TnpA-mediated cleavage and strand exchange activities of these engineered transposon sequences in vitro as previously described, which showed that these modified elements were as competent as the wild type element in performing all transposition steps in vitro, including LE and RE cleavage, generation of a RE-LE transposon junction and insertion of this junction into a target substrate.

To investigate target site specificity, the inventors analyzed the integration activities of engineered transposon junctions (Ji, with ‘i’ indicating a specific variant number) into complementary targets (Tic) in vitro on sequencing PAGE. 14 nM of 5′-labeled oligonucleotide (either IS6o8 LE, RE, RE-LE junction or target, as indicated) was incubated with 10 μM TnpA for 1 h at 37° C., in buffer containing 20 mMHEPES [pH 7.5], 160 mM NaCl, 5 mM MgCl₂, 10 mMDTT, 20 μg/ml BSA, 0.5 μg of poly-dIdC and 20% glycerol. For strand transfer reactions, additional unlabeled oligonucleotide substrates were added at 1 μM final concentration. Reactions were terminated by addition of 0.1% SDS and incubation for 15 min at 37° C. Products were heat-denatured, separated on a 10% sequencing TBE-Urea PAGE gel and analyzed by phosphorimaging on a Typhoon™ FLA 9500 (GE Healthcare Life Sciences). A 20/100 Oligo Length Standard (IDT) was radioactively labeled (5′-32P) as described above and loaded in every gel.

Several sequence pairs were tested and two representative examples, Ji/Tic and J2/T2c, are shown in FIG. 2C. In FIG. 2(C), sequencing DNA PAGE gel monitoring J1 cleavage and integration into its T1c complementary target is shown. A random target substrate containing a TTAC site but no additional complementarity to the junction (marked as Tr) was used in a competition reaction with T1c (in 1:1 molar ratio) to monitor integration specificity (lane 5). Tr contains a shorter (30 nt) 3′ segment following the cleavage site than Tic, so that the integration products can be clearly distinguished. Schematics for the labeled junction substrate (a), the cleavage product (d) and integration products with T1c (b) or Tr (c) are shown on the right. The modified transposon junctions integrated efficiently into their complementary target, as shown by the specific formation of strand transfer products between J1 and T1c in all cases (lane 3 in both FIG. 2C). Integration reactions including equimolar concentrations of the complementary target and a random target with no extra complementarity to the junction substrate beyond the canonical TTAC (CT), showed an explicit preference for integration into the complementary target (lane 5 in FIG. 2C). The preferential selection of targets with the extra complementarity region was also clearly observed in the presence of two different pools of random targets containing scrambled sequences in the 8 nt variable region (TsI and TsII; see FIG. 4B) at various Tic:Ts concentration ratios (FIG. 2D, lanes 6-15). FIG. 2(D) shows that J1 integrates predominantly into its complementary target (Tic), even in the presence of 20-fold molar excess of random target substrates. Competition assays with 2 different scrambled target pools (TsI and TsII), containing a conserved TTAC site and different sets of scrambled sequences in the 8 nt variable region, are shown. The molar ratio of T1c:TsI or T1c:TsII is indicated above the gel. Positions of the J1 substrate (a), cleavage (d) and strand transfer products with T1c (b) or TsI/TsII (c) in the sequencing gel are indicated by arrows.

### Example 3: Extended LE/Target Recognition can be Combined with Targeting of Altered CT Sequences

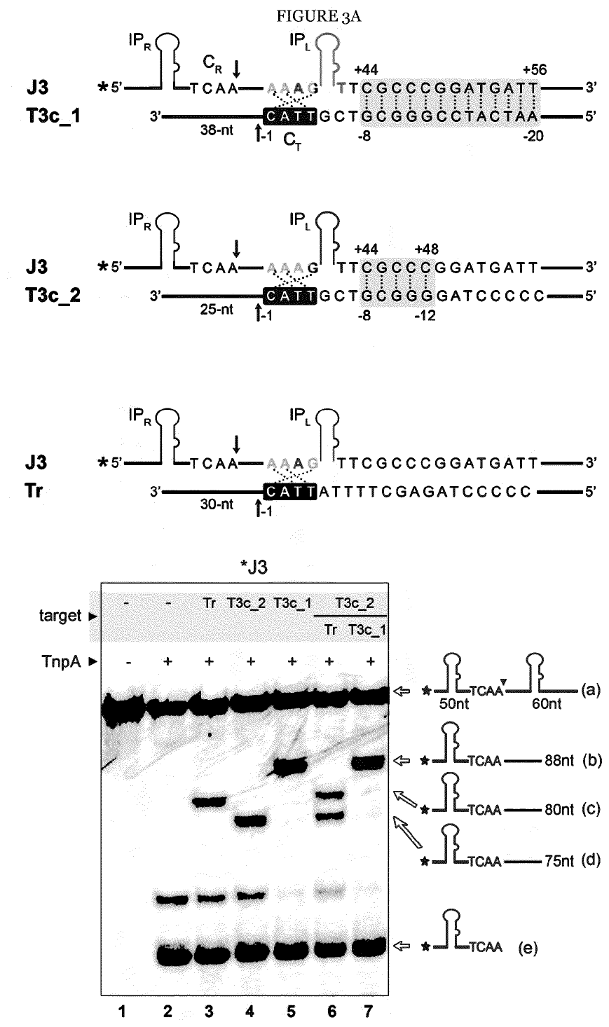

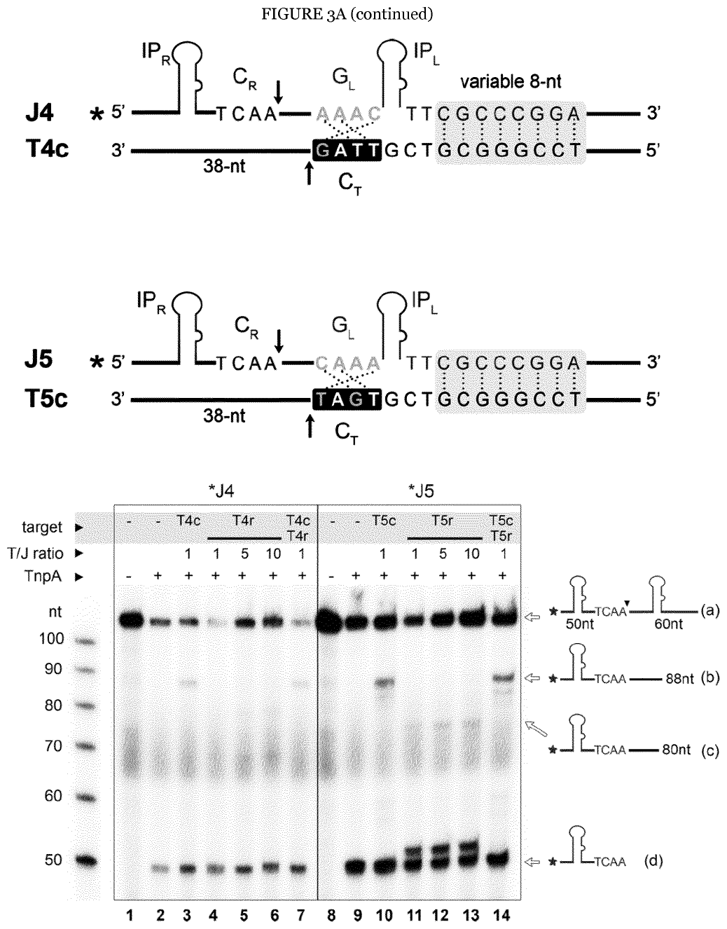

It was previously demonstrated that IS6o8 insertion can be redirected to alternative tetranucleotide target sequences by mutating the transposon guide sequence. Although engineered transposons were less efficient, they were very specific for integration into the intended sites. To further explore the scope of the targeting strategy of this invention, the inventors tested the ability of IS608 to select even more specific targets by further increasing the region of base complementarity. FIG. 3(A) shows representative data for a junction/target pair with a 13 bp complementary region in addition to the GL/CT interaction (see light blue shade in the scheme; J3/T3c 1). Integration of J3 to T3c 1 (lane 5) was compared with a target containing only 5 nt complementarity in the variable region (light blue; T3c 2) or a random target (Tr, which contains only GL/CT complementarity, lane 3) maintaining only the GL/CT interaction and to a target containing 5 complementary bases in addition to the CT site (T3c 2, lane 4). Target substrates contain various 3′ segments following the cleavage site to distinguish integration products. In competition reactions (lanes 6, 7), targets were combined in 1:1 molar ratio. Bands corresponding to the substrates and products are indicated on the right. Remarkably, while the 5 bp long complementarity did not enable efficient selection of T3c 2 over Tr (lane 6), integration was exclusively directed to T3c 1 in the presence of an equimolar amount of T3c 2 (lane 7). These results provide proof of concept for specific targeting of engineered IS6o8 transposons to selected 12-17 nt long sequences, with 4 nt defined by the native GL/CT interaction and extra 8-13 nt defined by engineered extended complementarity.

The inventors analyzed the potential of combining the previous CT resetting strategy with this new extended target recognition method for two different junction/target complementary pairs with mutations in GL and CT, J4/T4c and J5/T5c. FIG. 3(B) shows IS6o8 targeting to integration sites with alternative CT sequences. Light-blue shade highlights the complementary regions and arrow marks the cleavage positions. The engineered substrates contain the same 8 bp extended complementary region as Ji/Tic, but with one or two GL-CT base pairs also modified (FIG. 3B). The inventors assayed TnpA mediated cleavage and integration activity with these substrates on a sequencing gel, including competition reactions with random targets T4r and T5r as control (containing the same CT as in T4c and T5c, respectively, but without extended complementarity to LE, i.e. a random sequence in the 8 nt variable region). Integration products with J4/T4r and J5/T5r substrate pairs were not detected, even with 10-fold excess of the random target (FIG. 3B, lanes 4-6 and 11-13), in agreement with the previously observed decrease in activity with redirected GL/CT sites. T4r and T5r contain a shorter (30 nt) 3′ segment following the cleavage site. Interestingly, integration activity was greatly enhanced by introduction of the extra 8 bp complementary sequence in the engineered J4/T4c and J5/T5c pairs (lanes 3 and 10), indicating that extended base pairing with the target can rescue transposon integration. The extended complementary target sites were also preferentially chosen in competition experiments, compared with the random targets (FIG. 3B, lanes 7 and 14). Substrates and products are shown schematically on the right of FIG. 3B.

1. A polynucleotide comprising a nucleic acid sequence encoding for, or being, at least one transposon end, wherein said at least one transposon end has the following structure in 5′ to 3′ direction: a 5′ flanking region, a transposase recognition site, such as a 5′ guide sequence, optionally a cloning cassette for introducing a genetic cargo construct, and a 3′ flanking region; characterized in that the 5′ flanking region and/or the 3′ flanking region comprise a target complementarity region having a sequence identity of at least 60% to a nucleic acid sequence in a target genome.

2. The polynucleotide according to claim 1, wherein the polynucleotide further comprises in 5′ to 3′direction:
(a) Optionally, a 5′-cleavage site,
(b) a 5′-guide sequence,
(c) one or more 5′—transposon structural element(s),
(d) optionally, the cloning cassette for introducing a genetic cargo construct,
(e) a 3′ guide sequence,
(f) One or more 3′ transposon structural element(s),
(g) Optionally, a 3′-cleavage site.

3. The polynucleotide according to claim 2, wherein the target complementarity region is in close sequence proximity to the 5′-guide sequence, preferably not more than 100 nucleotides apart, more preferably not more than 50 nucleotides apart, most preferably between 15 to 40 (or 20 to 30) nucleotides apart from the 3′end of 5′ guide sequence.

4. The polynucleotide according to claim 2 or 3, wherein the target complementarity region is located 3′ of the first 5′ transposon structural element.

5. The polynucleotide according to any one of claims 2 to 4, wherein the 5′ and/or 3′ transposon structural element is a sequence, that is capable of forming a 3-dimensional structure, such as a hairpin, within the polynucleotide and preferably is selected from imperfect or perfect palindrome sequences, inverted terminal repeats (ITRs), and/or direct terminal repeats (DTRs), most preferably wherein said transposon structural element is an imperfect palindrome sequence.

6. The polynucleotide according to any one of claims 2 to 5, wherein
(a) The 5′-cleavage site has the sequence TTAC; and/or
(b) The 3′-cleavage site has the sequence TCAA; and/or
(c) The 5′-guide sequence has the sequence AAAG; and/or
(d) The 3′-guide sequence has the sequence GAAT; and/or
optionally, with not more than one, preferably none, nucleotide variation of these sequences.

7. The polynucleotide according to any one of claims 1 to 6, wherein the polynucleotide does not comprise a sequence encoding for a functional transposase protein, preferably not comprising a tnpA and/or tnpB gene.

8. The polynucleotide according to any one of claims 1 to 7, wherein the cloning cassette comprises one or more restriction enzyme recognition sites.

9. The polynucleotide according to any one of claims 1 to 8, wherein the target complementarity region comprises a sequence complementary to a region within a target genome, which (i) is located outside of an expressible genetic element, or (ii) within an expressible genetic element, and preferably wherein (ii) the target complementarity region is selected such that the expressible genetic element is not expressible, or reduced expressible, or increased expressible and/or the expressed sequence is dis-functional after integration of the transposon.

10. The polynucleotide according to any one of claims 1 to 9, wherein the transposon is not capable of being mobilized within a target genome.

11. The polynucleotide according to any one of claims 1 to 10, wherein said polynucleotide is RNA, DNA, cDNA, PNA, or a combination thereof.

12. The polynucleotide according to any one claims 1 to 11, wherein said transposase recognition site comprises one or more transposon structural elements and comprises imperfect palindrome sequences, inverted terminal repeats (ITRs), and/or direct terminal repeats (DTRs), preferably wherein said transposase recognition site comprises imperfect palindrome sequences.

13. The polynucleotide according to any one of claims 1 to 12, wherein said transposon is a single-strand transposon, and/or a double-strand transposon, preferably wherein said transposon is a single-strand transposon.

14. The polynucleotide according to any one of claims 1 to 13, wherein said target complementarity region is 1 to 100 nucleotides long, more preferably 2 to 50, more preferably 4 to 20, and most preferably 8 to 13 nucleotides long.

15. The polynucleotide according to any one of claims 1 to 14, wherein no nucleic acid sequence is located between said target complementarity region and said transposase recognition site or wherein a nucleic acid sequence is located between said target complementarity region and said transposase recognition site, preferably wherein said sequence is between 1 and 100 nucleotides long, more preferably between 1 and 10 nucleotides long, even more preferably between 1 and 5 nucleotides long, and most preferably wherein said sequence is 2 or 3 nucleotides long.

16. The polynucleotide according to any one of claims 1 to 15, wherein said target complementarity region is located in the 3′ flanking region, preferably wherein said target complementarity region is located at the 5′ end of said 3′ flanking region, or wherein said target complementarity region is located in the 5′ flanking region, preferably wherein said target complementarity region is located at the 3′ end of said 5′ flanking region.

17. The polynucleotide according to any one of claims 1 to 16, wherein said polynucleotide is for site-specific insertion of a nucleotide sequence of interest to be inserted into the genome of a target cell, into a target plasmid, and/or into a target polynucleotide, preferably wherein said nucleotide sequence of interest is a modified transposon sequence, optionally wherein the transposase gene is replaced with a sequence of interest.

18. The polynucleotide according to any one of claims 1 to 17, wherein said transposon sequence comprises an insertion sequence (IS) of the IS200/IS605 family, such as IS608, ISDra2, IS605, or of IS630, IS701, IS607, IS982, IS3, IS1, IS6, IS5, IS4, or IS66, preferably wherein said insertion sequence (IS) is IS608.

19. The polynucleotide according to any one of claims 1 to 18, wherein said transposase recognition site comprises the nucleic acid sequence of SEQ ID No: 61 (CCCCTAGCTITTAGCTATGGGG).

20. A composition comprising the polynucleotide according to any one of claims 1 to 19, and a nucleic acid encoding an integrating enzyme, optionally wherein said nucleic acid is under the control of a promoter element, or an integrating polypeptide, such as an integrating enzyme polypeptide.

21. The composition according to claim 20, wherein the nucleic acid is DNA, cDNA, PNA, RNA, or a combination thereof, or an expression vector expressing said nucleic acid.

22. The composition according to claim 20 or 21, wherein the polynucleotide encoding for said transposon and the nucleic acid encoding said integrating enzyme are the same nucleic acid molecules.

23. The composition according to claim 20 or 21, wherein the polynucleotide encoding for said transposon and the nucleic acid encoding said integrating enzyme are separate nucleic acid molecules.

24. The composition according to any one of claims 20 to 23, wherein said integrating enzyme is a transposase of the IS200/IS605 family, such as IS608, or a IS630, IS701, IS607, IS982, IS3, IS1, IS6, IS5, IS4, or IS66 transposase.

25. The composition according to any one of claims 20 to 24, wherein said integrating enzyme is a histidine-hydrophobic-histidine endonuclease, such as the IS6o8-encoded transposase TnpA.

26. An expression construct, comprising an expressible polynucleotide according to any one of claims 1 to 19, or a composition according to any one of claims 20 to 25, and a promoter element, wherein the promoter element is operably linked to the expressible polynucleotide to allow for the expression of the polynucleotide.

27. A nucleic acid vector comprising a polynucleotide according to any one of claims 1 to 19, a composition according to any one of claims 20 to 25, and/or an expression construct according to claim 26.

28. A recombinant cell comprising a polynucleotide according to any one of claims 1 to 19, a composition according to any one of claims 20 to 25, an expression construct according to claim 26, and/or a nucleic acid vector according to claim 27.

29. A transposon system comprising
(a) a polynucleotide according to any one of claims 1 to 19, an expression construct according to claim 26, a nucleic acid vector according to claim 27, and/or a recombinant cell according to claim 28; and
(b) a transposase polypeptide of the IS200/IS605 family, such as IS6o8, or a IS630, IS701, IS607, IS982, IS3, IS1, IS6, IS5, IS4, or IS66, preferably wherein said transposase polypeptide is the IS6o8-encoded transposase TnpA.

30. In vitro use of a transposon system according to claim 29 for gene delivery into a target cell, a target plasmid, and/or a target polynucleotide.

31. An in vitro method for gene delivery into a target cell comprising the following steps:
(a) bringing into contact the transposon system according to claim 29 with a target cell; and
(b) culturing said target cell under conditions permissive to the culture of said target cell.

32. A pharmaceutical composition comprising a polynucleotide according to any one of claims 1 to 19, a composition according to any one of claims 20 to 25, an expression construct according to claim 26, a nucleic acid vector according to claim 27, a recombinant cell according to claim 28, and/or a transposon system according to claim 29, together with a pharmaceutically acceptable carrier and/or excipient.

33. A kit comprising
(a) a polynucleotide according to any one of claims 1 to 19, an expression construct according to claim 26, a nucleic acid vector according to claim 27, and/or a recombinant cell according to claim 28; and
(b) a transposase polypeptide of the IS200/IS605 family, such as IS6o8, or a IS630, IS701, IS607, IS982, IS3, IS1, IS6, IS5, IS4, or IS66 transposase, preferably wherein said transposase polypeptide is the IS6o8-encoded transposase TnpA.

34. A kit comprising a polynucleotide according to any one of claims 1 to 19, a composition according to any one of claims 20 to 25, an expression construct according to claim 26, a nucleic acid vector according to claim 27, a recombinant cell according to claim 28, or a transposon system according to claim 29; and instructions for use.

35. A compound for use in the treatment of a disease, wherein the compound is selected from the polynucleotide according to any one of claims 1 to 19, the composition according to any one of claims 20 to 25, the expression construct according to claim 26, the nucleic acid vector according to claim 27, the recombinant cell according to claim 28, the transposon system according to claim 29, the pharmaceutical composition according to claim 32, and/or the kit according to claim 33 or 34, preferably wherein the disease is an infectious and/or a proliferative disease, more preferably wherein said infectious disease is a bacterial infection, such as an H. pylori infection, and/or wherein said proliferative disease is cancer, such as non-small cell lung cancer, small cell lung cancer, renal cell cancer, brain cancer, gastric cancer, colorectal cancer, hepatocellular cancer, head and neck cancer, pancreatic cancer, prostate cancer, leukemia, breast cancer, Merkel cell carcinoma, melanoma, ovarian cancer, urinary bladder cancer, uterine cancer, gallbladder and bile duct cancer, esophageal cancer, or a combination thereof, preferably wherein said cancer is H. pylori-induced cancer.

36. The polynucleotide according to any one of claims 1 to 19, the composition according to any one of claims 20 to 25, the expression construct according to claim 26, the nucleic acid vector according to claim 27, the recombinant cell according to claim 28, the transposon system according to claim 29, the pharmaceutical composition according to claim 32, and/or the kit according to claim 33 or 34, for use in the diagnosis and/or treatment of an infectious and/or proliferative disease, or for use in the manufacture of a medicament against an infectious and/or proliferative disease, preferably wherein said infectious disease is a bacterial infection, such as an H. pylori infection, or wherein said proliferative disease is cancer, such as non-small cell lung cancer, small cell lung cancer, renal cell cancer, brain cancer, gastric cancer, colorectal cancer, hepatocellular cancer, head and neck cancer, pancreatic cancer, prostate cancer, leukemia, breast cancer, Merkel cell carcinoma, melanoma, ovarian cancer, urinary bladder cancer, uterine cancer, gallbladder and bile duct cancer, esophageal cancer, or a combination thereof, preferably wherein said cancer is H. pylori-induced cancer.

37. A method of preventing and/or treating an infectious and/or a proliferative disease in a subject, the method comprising administering to the subject an effective amount of a polynucleotide according to any one of claims 1 to 19, the composition according to any one of claims 20 to 25, the expression construct according to claim 26, the nucleic acid vector according to claim 27, the recombinant cell according to claim 28, the transposon system according to claim 29, the pharmaceutical composition according to claim 32, thereby preventing and/or treating said infectious and/or proliferative disease in the subject, preferably wherein said infectious disease is a bacterial infection, such as an H. pylori infection, or wherein said proliferative disease is cancer, such as non-small cell lung cancer, small cell lung cancer, renal cell cancer, brain cancer, gastric cancer, colorectal cancer, hepatocellular cancer, head and neck cancer, pancreatic cancer, prostate cancer, leukemia, breast cancer, Merkel cell carcinoma, melanoma, ovarian cancer, urinary bladder cancer, uterine cancer, gallbladder and bile duct cancer, esophageal cancer, or a combination thereof, preferably wherein said cancer is H. pylori-induced cancer.

38. The polynucleotide according to any one of claims 1 to 19, the composition according to any one of claims 20 to 25, the expression construct according to claim 26, the nucleic acid vector according to claim 27, the recombinant cell according to claim 28, the transposon system according to claim 29, the pharmaceutical composition according to claim 32, and/or the kit according to claim 33 or 34, for use in a gene delivery method, preferably wherein said method is for gene delivery into a cell selected from an eukaryotic and a prokaryotic cell, such as a cell of a human, a mouse, a rabbit, a dog, a monkey, a cat, a bacterium, or a yeast cell.

39. The polynucleotide according to any one of claims 1 to 19, the composition according to any one of claims 20 to 25, the expression construct according to claim 26, the nucleic acid vector according to claim 27, the recombinant cell according to claim 28, the transposon system according to claim 29, the pharmaceutical composition according to claim 32, and/or the kit according to claim 33 or 34, for use in manufacturing an engineered cell for a cell therapy, preferably wherein said cell is a T cell or a T cell progenitor, such as a CAR-T cell.

40. The method according to claim 39, wherein the T cell or T cell progenitor is autologous, or wherein the T cell or T cell progenitor is allogeneic.

41. A method of treating cancer in a subject in need thereof, comprising:
(a) isolating a cell from said subject or from a healthy donor;
(b) transfecting or transforming the cell with a polynucleotide according to any one of claims 1 to 19, the composition according to any one of claims 20 to 25, the expression construct according to claim 26, the nucleic acid vector according to claim 27, the recombinant cell according to claim 28, the transposon system according to claim 29, to produce a transfected or transformed cell;
(c) expanding the transfected or transformed cell to produce a plurality of transfected or transformed cells; and
(d) administering the plurality of transfected or transformed cells to said subject.

42. The method of claim 41, wherein the cell is a T cell or a T cell progenitor, optionally wherein said T cell or T cell progenitor is autologous or allogeneic.
